(19) United States
(12) Patent Application Publication
(10) Pub. No.: US 2008/0139885 A1
(43) Pub. Date: Jun. 12, 2008

(54) Autoclavable antireflective coatings for endoscopy windows and related methods

(76) Inventor: Jamie Knapp, Mendon, MA (US)

Correspondence Address: Brian F. Swienton;Newport Corporation, 1791 Deere Avenue, Irvine, CA, 92606, US

(21) Appl. No.: 11/982,310

(22) Filed: Oct. 31, 2007

Related U.S. Application Data
(60) Provisional application No. 60/859,688, filed on Nov. 16, 2006.

(51) Int. Cl. A61B 1/00; A61L 31/00

(52) U.S. Cl. 600/133; 600/176; 427/2.12

(57) ABSTRACT

The present application discloses various embodiments of optical windows for use within an endoscope and includes a substrate sized to be coupled to the endoscope and defining a first surface and at least a second surface, and at least one autoclavable coating applied to at least one of the first surface and second surface.

### CROSS-REFERENCE TO RELATED APPLICATIONS

The present application claims priority to U.S. Provisional Patent Application Ser. No. 60/859,688, filed Nov. 16, 2006, the entire contents of which are hereby incorporated by reference in its entirety herein.

### BACKGROUND

Sterilization of medical devices and instruments has been proven to greatly reduce the risk of post-operative infection. Sterilization of these medical devices may be accomplished in any variety of ways. Commonly, a medical autoclave is employed to subject contaminated devices and fixtures to a high temperature pressurized steam environment, thereby sterilizing these devices. Autoclaves generally consist of a sealed high-pressure vessel, which allows steam to enter at elevated pressure (typically about 15 psi or greater). The temperature within the autoclave is heated to a temperature of about 121 degrees Celsius or greater, the critical temperature at which biological contamination is optimally killed. A typical sterilization cycle consists of exposing a medical object to this high temperature condition for at least 15 minutes.

Endoscopy is a minimally invasive diagnostic medical procedure employed to evaluate the interior surfaces of an organ by inserting a small scope into the body. As such, endoscopes undergo a sterilization process, typically autoclaving, before use. As shown in FIG. 1, endoscope systems 1 typically employ a light source 3 and a handpiece 7 coupled to the light source 3 via a conduit 5. The handpiece 7 may include an elongated body 9 configured to deliver the light to and from an area of interest within the body. Fiber optic devices are often employed within the conduit 5 and handpiece 7. As such, the conduit 5 and at least a portion of the handpiece 7 may be flexible or rigid. In addition, one or more cameras or viewing devices may be included within the endoscope device 1 or externally coupled thereto. Typically, the handpiece 7 includes at least one optical window 11 configured to seal the interior of the handpiece 7 without adversely affecting the optical characteristics thereof. While any variety of materials may be used to manufacture the optical window 11, sapphire optical windows are most often used in endoscopes due to its superior durability over other materials. However, sapphire possesses undesirable optical characteristics at some wavelengths. For example, the high index of refraction of sapphire (typically about 1.787 at about 400 nm and about 1.760 at about 750 nm) causes undesirable high optical reflective losses at the surfaces of the sapphire window, which are typically greater than about 8% per surface (See FIG. 2). As such, the transmission of a sapphire window of only about 84% (See FIG. 3).

Thus, in light of the foregoing, there is an ongoing need for autoclavable optical coatings for endoscope windows having lower surface reflectance then uncoated endoscope windows. Further, there is an ongoing need for a process for applying autoclavable optical coatings to endoscope windows.

### SUMMARY

Various embodiments of autoclavable coated endoscope windows are disclosed herein. In one embodiment, the present application discloses an optical window for use within an endoscope and includes a substrate sized to be coupled to the endoscope and defining a first surface and at least a second surface, and at least one coating applied to at least one of the first surface and second surface.

In another embodiment, the present application is directed to an endoscope window configured to withstand multiple autoclaving processes and includes a sapphire substrate sized to coupled to an endoscopy handpiece and defining a first surface and at least a second surface, at least one first surface coating layer applied to the first surface, and at least one second surface coating applied to the second surface.

In addition, the present application discloses a method of producing a coated endoscope window configured to withstand multiple autoclaving processes and includes positioning one or more endoscope window substrates within an evacuatable coating vessel, inserting at least one coating material into at least one containment structure positioned located within the coating vessel, evacuating the containment vessel to a about pressure of about 3×10−6 mbar or less, activating one or more intense electron beams into the containment structure, vaporizing the coating material at about room temperature or greater with the electron beam, and depositing the vaporized coating material onto the one or more endoscope windows.

In another embodiment, the present application is directed to a method of producing a coated endoscope window configured to withstand multiple autoclaving processes and includes positioning one or more endoscope window substrates within an evacuatable coating vessel having one or more coating materials located therein, inserting at least one coating material into at least one containment structure positioned located within the coating vessel, and depositing the vaporized coating material onto the one or more endoscope windows at room temperature using an ion plating process.

Other features and advantages of the embodiments of the endoscope windows having autoclavable coatings applied thereto as disclosed herein will become apparent from a consideration of the following detailed description.

### BRIEF DESCRIPTION OF THE DRAWINGS

Various endoscope windows having autoclavable coatings applied thereto will be explained in more detail by way of the accompanying drawings, wherein

FIG. 1 shows a schematic diagram of an embodiment of a prior art endoscope system having an optical window coupled to a handpiece configured to be inserted into a body;

FIG. 2 shows a graphical representation of a reflective spectral property of a current, state-of-the-art uncoated sapphire endoscope window;

FIG. 3 shows a graphical representation of a transmissive spectral property of a current, state-of-the-art uncoated sapphire endoscope window;

FIG. 4A shows a perspective view of an embodiment of an optical window having at least one coating applied to a first surface thereof and configured to withstand multiple autoclaving processes;

FIG. 4B shows a perspective view of an embodiment of an optical window having at least one coating applied to a first and second surface thereof and configured to withstand multiple autoclaving processes;

FIG. 5 shows a graphical representation of a reflective spectral property of an embodiment of a coated endoscope window;

FIG. 6 shows a graphical representation of a transmissive spectral property of an embodiment of a coated endoscope window; and

FIG. 7 shows a schematic diagram of an ion plating coating apparatus configured for coating endoscope windows.

### DETAILED DESCRIPTION

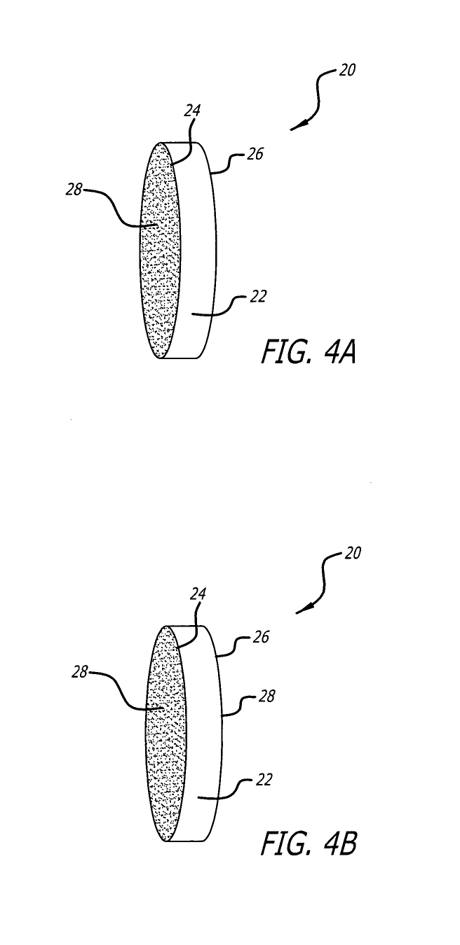

FIGS. 4A and 4B shows perspective views of embodiments of an optical window for use within an endoscope system or device. As shown, the optical window 20 comprises a body or substrate 22 defining a first surface 24 and at least a second surface 26. In the illustrated embodiments, the first and second surfaces 24, 26 are substantially planar and parallel. In an alternate embodiment, at least one of the first surface 24 and the second surface 26 may be substantially non-planar. Further, the first surface 24 and second surface 26 need not be parallel. The substrate 22 of the optical window 20 may be sized to be coupled to or otherwise retained within an endoscope device. Those skilled in the art will appreciate that the optical window 20 may be used in any variety of devices requiring autoclaving and/or steam-based sterilization other than endoscopes. As such, the optical window 20 may be manufactured in any variety of sizes and dimensions. Further, the optical window 28 disclosed herein may be used in any variety of devices used in steam and/or high heat/high humidity environments. As such, the coated optical window 20 disclsoed herein may be used in any variety of industries. The optical window 20 may be manufactured from any variety of materials. For example, the optical window 20 may be manufactured from sapphire. In the alternative, the optical window 20 may be any variety of materials, including, without limitation, doped-sapphire, Al₂O₃ based materials, fused silica, glass, composite materials, silica, and the like.

Referring again to FIGS. 4A and 4B, at least one of the first surface 24, the second surface 26, or both include at least one optical coating layer 28 applied thereto. In the embodiment shown in FIG. 4A, the first surface 24 of the window 20 includes a coating layer 28 thereon, while FIG. 4B shows an embodiment wherein the coating layer 28 is applied to the first and second surfaces 24, 26 of the window 20. The coating layer 28 may comprise a single layer of a material, or, in the alternative, multiple layers of one or more materials. Any variety of materials may be used to for the coating layer 28. For example, the coating layer 28 may include, without limitation, various metallic oxides, silicon dioxide (SiO₂), aluminum oxide (Al2O5), Hafnium Oxide (HfO2), Tantulum Pentoxide (Ta2O5), anti-reflective coatings, bandpass filter coatings, wavelength selective coatings, protective overcoats, and the like. For example, FIG. 4A shows an embodiment of a coated optical window 20 comprising one or more thin film layers 28 of Hafnium Oxide applied to the first surface 24.

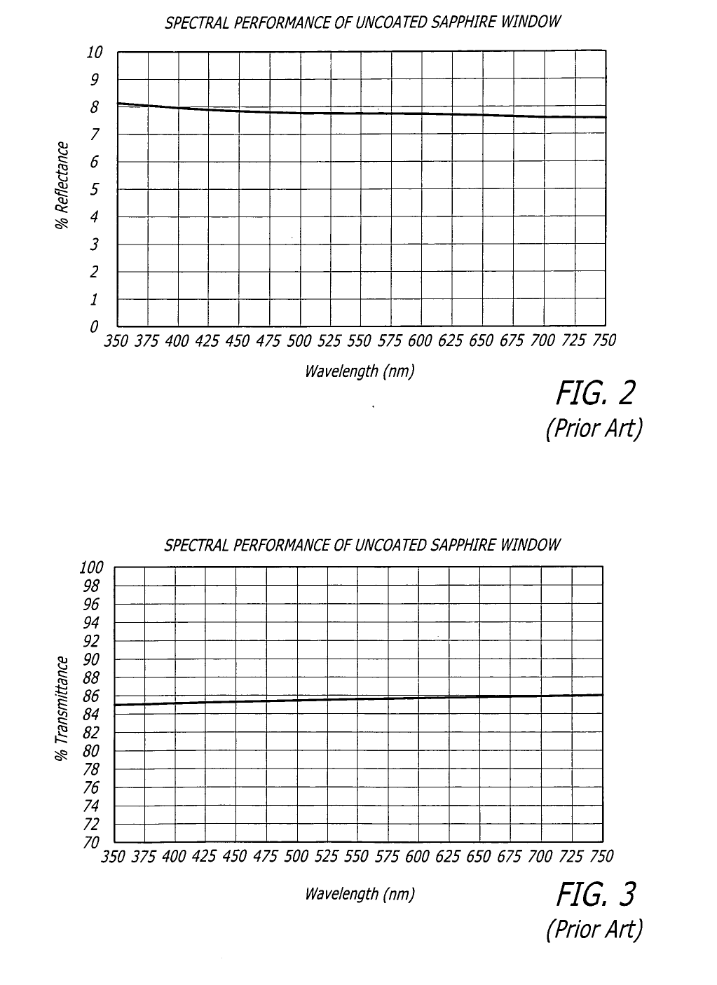

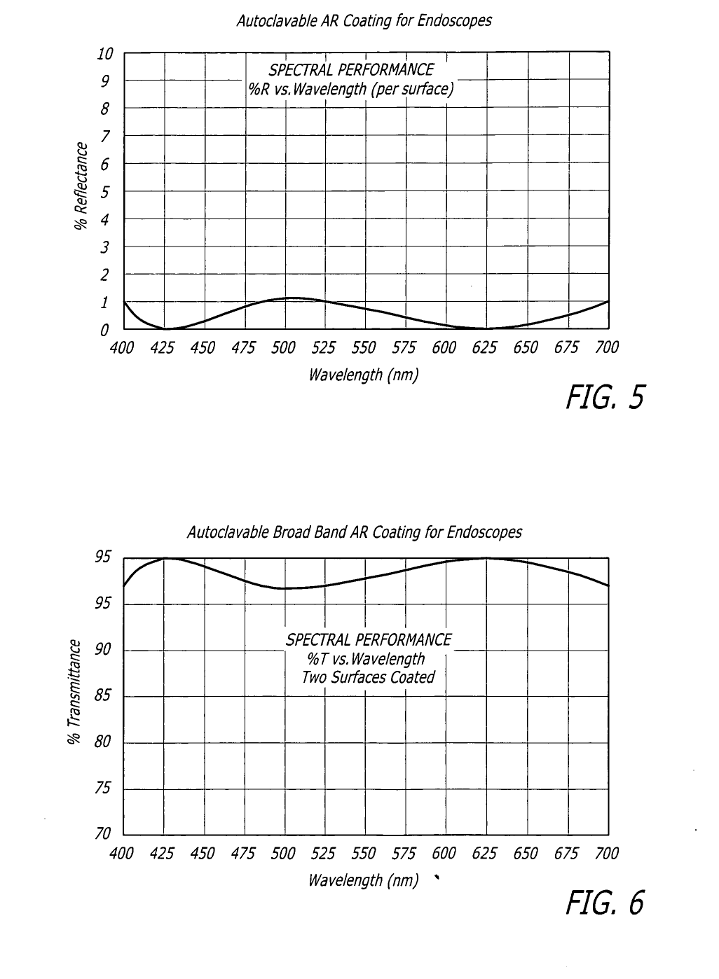

FIGS. 5 and 6 show a graphical representation of the performance of the coated optical window shown in FIG. 4B. As stated above, one or more thin film layers 28 of Silicon and Hafnium Oxide where applied to a sapphire substrate 22. As shown in FIG. 5, Silicon and Hafnium Oxide forming the coating layer 28 acts as an anti-reflective coating thereby decreasing light reflected by the first surface 24 and second surface 26 as compared with an uncoated sapphire optical window (See. FIG. 2). Optionally, the coating layers 28 may be applied to the first surface 24, second surface 26, or both the first and second surfaces 24, 26 of the substrate 22. As shown in FIG. 6, the transmittance of the coated optical window of FIG. 4B is greater than the transmittance of an uncoated optical window (See FIG. 3).

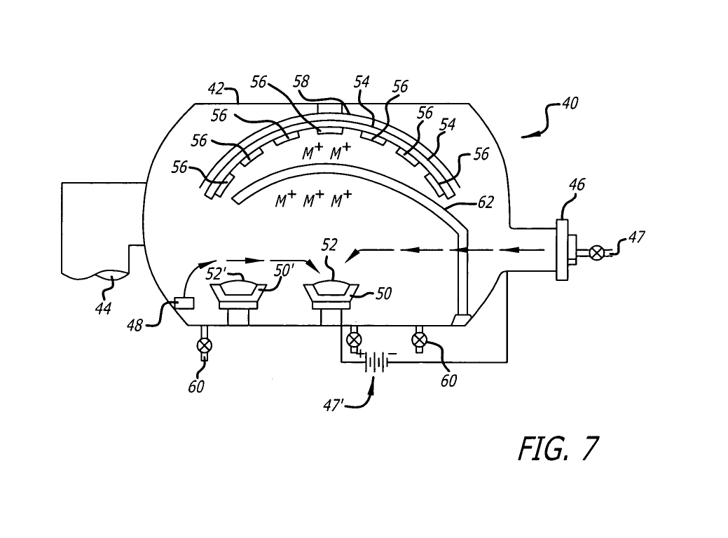

The layers of autoclavable optical coatings may be applied to optical windows in any variety of ways. For example, FIG. 7 illustrates an exemplary ion plating coating apparatus 10 as described in U.S. Pat. No. 6,139,968, the entirely of which is incorporated by reference herein. As shown, the coating apparatus 40 includes an evacuatable coating vessel 42 and an evacuation device 44 in fluid communication with the vessel 42. As such, the evacuation device 44 is configured to remove fluid from and/or provide fluid to the vessel 42. At least one deposition plasma source 46 and one or more electron beam guns 48 configured to supply electrons of energy directed towards one or more containment structures 50, 50′ are positioned within the vessel 42. In the illustrated embodiment, two 270° electron beam guns are positioned within the vessel 42, although those skilled in the art will appreciate that any number of type of electron beam guns may be used. The deposition plasma source 46 may include a heated tantalum filament or other heating device and a gas inlet 47.

As shown in FIG. 7, two containment structures 50, 50′ are positioned within the vessel 42. In one embodiment the containment structures 50, 50′ may comprise an electrically conductive structure 50, 50′ that may be coupled to the plasma source 46 via at least one low voltage, high current power supply 47′. Those skilled in the art will appreciate that any number of containment vessels may be positioned within the vessel 42. The number of containment structures 50, 50′ within the vessel 42 may vary depending on the composition of the coating layer(s) to be produced by the apparatus 40. For example, the first crucible 50 holds a first source material (e.g., a titanium source material), while the second crucible holds a second source material (e.g., a silicon source material). As such, the separate source chemicals will be separately activated by one or more electron guns 48. Those skilled in the art will appreciate that the first and second crucibles 50, 50′ may be configured to hold the same or different materials.

Further, the containment vessels 50, 50′ may be constructed from any variety and combination of materials, including, without limitation, copper crucibles, molybdenum, stainless steel, aluminum, gold, silver, titanium, various metals, glass, ceramics, composite materials, polymers, and the like. The containment structures 50, 50′ are configured to receive on or more coating materials 52 and 52′. Exemplary coating materials 52, 52′ include, without limitation, various metallic oxides, silicon dioxide (SiO₂), aluminum oxide (Al₂O₅), Hafnium Oxide (HfO₂), Tantulum Pentoxide (Ta₂O₅), silicon, titanium, aluminum, tantalum, hafnium, zirconium, anti-reflective coatings, bandpass filter coatings, wavelength selective coatings, protective overcoats, and the like. In one embodiment, the first and second surfaces 24, 26 are coated with the same coating material. In an alternate embodiment, the first and second surfaces 24, 26 are coated with different coating materials. An exemplary suitable coating apparatus 40 is the BAP 800 Batch Ion Plating System, which is commercially available from Balzers Aktiengesellschaft of Liechtenstein, although any variety of systems may be used.

Referring again to FIG. 7, the coating apparatus 40 further includes at least one substrate support structure 54 positioned within the vessel 42. In the illustrated embodiment, the substrate support structure 54 is positioned opposite the containment structures 50, 50′ and configured to support one or more substrates 56 onto which the coating materials 52, 52′ are to be deposited/applied as coating layers. Optionally, the substrate support structure 54 may be formed in any variety of shapes and/or configurations, including, without limitation, an electrically isolated substrate support structure, a rotatable substrate support structure, a dome-shaped structure, and the like. Further, substrate support structure 54 may be coupled to any surface of the vessel 42.

Any number and variety of substrates 56 may be positioned within the vessel 42 and coated. Exemplary substrates 56 include, without limitation, sapphire substrates, doped-sapphire substrates, Al₂O₃ based substrates, fused silica substrates, glass substrates, composite optical substrates, silica substrates, metal substrates, plastic substrates, semiconductor substrates, and electronic device substrates, substrates manufactured from crown glass, soda-lime float glass, natural quartz, synthetic fused silica, Schott BK-7, and the like.

As shown in FIG. 7, one or more feedlines 60 may be in fluid communication with the vessel 42 and configured to provide one or more fluids thereto. Exemplary fluids include, without limitation, reactive gases and the like. In one embodiment, the reactive gases may be introduced into the vessel 42 through the feedlines 60 during deposition process. Further, one or more plasma sources 62 may be positioned within the vessel 42. The gas plasma sources 62 may be configured to introduce a pre-treatment gas such as oxygen, argon or nitrogen into the coating vessel 42.

During use, the coating vessel 42 is evacuated by vacuum system 44 to provide a base vacuum pressure to the coating vessel 42 of less than about 3×10−6 mbar. Thereafter, one or more electron beam guns 48 of deposition plasma source 46 direct one or more intense electron beams into the containment structure(s) 50, 50′, thereby vaporizing at least one of the coating material(s) 52 and 52′ contained therein. In one embodiment, multiple coating materials 52, 52′ may be applied to the substrates 56 sequentially. In another embodiment, multiple coating materials 52, 52′ are applied to the substrates 56 simultaneously.

The substrates 56 positioned on the substrate support structure 54 become negatively biased due to the deposition plasma discharge during the coating process. As a result, the vaporized coating material(s) (denoted by M+ in FIG. 7) activated by the deposition plasma becomes highly energetic, ionized and chemically reactive. The energized material M+ is attracted to the one or more substrates 56 via electromagnetic coulomb attraction, after which coating/film deposition occurs. It should be noted, however, that the deposition plasma procedure may be commenced immediately after the gas plasma pretreatment is completed, without vacuum interruption.

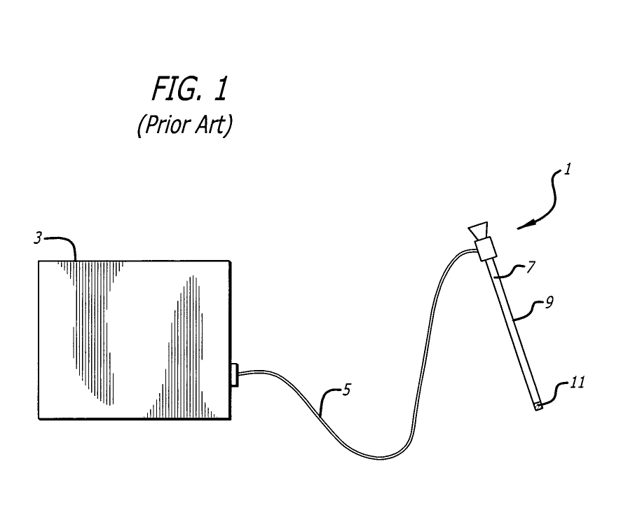

Unlike other coating processes known in the art, one or more autoclavable coatings layers 28 may be applied to the substrate 22 at about room temperature (See FIG. 4). As such, the substrates 56 need not be heated to a temperature greater than room temperature. As such, one or more exterior surfaces of an optical window 11 mounted within an endoscope device 1 may be coated using the method disclosed herein without requiring the window 11 to be removed from the handpiece 7 (See FIG. 1). Further, the coating apparatus 40 may further include one or more additional auxiliary devices (e.g., auxiliary coils for the production of magnetic fields, etc.), which are generally known in the art.

One or more reactive gases may be introduced into the vessel 42 prior to, during, or following the deposition process via one or more feedlines 60. For example, the feedlines 60 may be configured to discharge one or more reactive gases at a position proximate to the containment structures 50, 50′, thereby permitting the effective density of reactive gas to mix and react with material vaporized from the containment structure(s) 52, 52′ during the ion plating coating process. Any variety of reactive gases may be used, including, without limitation, oxygen, nitrogen, aliphatic and aromatic hydrocarbons (e.g., acetylene, methane, ethane, propylene, benzene, etc.) and/or similar reactive gases. For example, when depositing a coating that is comprised of titanium oxide, silicon dioxide, aluminum oxide and/or other oxygen-containing layers, oxygen may be supplied through one or more feedlines 60 to react with the one or more source chemicals/metals that are vaporized from containment structure 50 and/or 50′. Optionally, a mixture of one or more reactive gases may be introduced into coating vessel 42 to produce a coating layer of a desired composition onto the one or more substrate(s) 56. For example, nitrogen and acetylene may be simultaneously supplied through separate lines 60 to provide a carbonitride-type coating on the substrate(s) 56. Coating layers having other compositions also may be applied, as will be appreciated by those of ordinary skill in the art.

### EXAMPLE

A two-layer ion-plated Silicon Dioxide/Hafnium Oxide coating having a total physical thickness of about 202.3 nm was uniformly deposited at room temperature upon sapphire endoscope windows. The sapphire windows had a transverse dimension of about 18 mm and a thickness of about 1 mm. The particular design of this antireflective coating is:
AIR/SAPPHIRE WINDOW/116.8 nm H/85.5 nm L/AIR
where H refers to Hafnium Oxide and L refers to Silicon Dioxide

As will be appreciated by those skilled in the art, an alternative antireflective coating design (having spectral properties equivalent to the current example) incorporating a durable sapphire outer layer is:
AIR/SAPPHIRE WINDOW/116.8 nm H/81.7 nm L/1.88 M/AIR
where H refers to Hafnium Oxide, L refers to Silicon Dioxide and M refers to Aluminum Oxide.

As previously evaluated by scanning electron microscopy, the resultant glass-like coatings have an amorphous, fully densified physical structure, which mimic the optical, physical and chemical characteristics of the corresponding bulk materials.

Thereafter, the coated samples were subjected to multiple standard high-pressure steam sterilization processes (autoclaving) without signs of spectral or physical degradation. In one instance, a coated sample was subjected to over one hundred autoclaving processes without suffering an appreciable degradation performance.

In contrast, samples of antireflective coatings were deposited upon sapphire substrates using the current state-of-the-art ion-assisted magnetron sputtering processes. The design of this coating was:
AIR/SAPPHIRE WINDOW/113 nm H/88.2 nm L/AIR
where H refers to sputtered Tantalum Pentoxide and L refers to Silicon Dioxide

The ion-assisted magnetron sputtered samples were subjected to the same autoclave environment as described above. In this case, the optical coating became visibly stained and opaque by the absorption of moisture after 4 cycles. Delamination (film peeling) occurred after 9 cycles.

The ion plating deposition conditions for application of such a multilayer optical coating may generally vary within a range of values, and may be readily determined empirically based on the present disclosure. More specifically, for application of at least one thin film coating layer of SiO₂ onto a substrate, silicon is loaded into copper crucible containment structure 50′ of coating vessel 42 (See FIG. 7). Optionally, a pre-treatment plasma gas of oxygen or argon may be used. The gas plasma source 62 is used to provide a pre-treatment plasma step voltage of about 4.5 kV, a current of about 350 mA, and a duration of glow of about 30 to about 45 minutes. Following such a pre-treatment step, or directly after applying the desired vacuum to vessel 42 if a pre-treatment step is not carried out, the deposition plasma gas pressure within plasma source is about 2.8 mbar, the plasma voltage is in the range of about 55 to about 60 volts, the plasma current is in the range of about 55 to about 60 amps, the anode-to-ground voltage is about 40 volts, the plasma filament current is about 110 amps, the reactive gas is oxygen (introduced through feedline(s) 60 in FIG. 7), and the reactive gas pressure is about 1×10⁻³ mbar within the coating vessel 42. The electron beam gun(s) 48 for reagent evaporation can be operated at a high voltage of about 10 kV, an emission of about 400 mA and at a rate of about 0.5 nm/second.

Thus, the following conditions/parameters represent one embodiment for depositing a coating of silicon dioxide onto a substrate. Those skilled in the art will appreciate that at least one of these parameters may be altered by the user as desired. Further, deposition conditions/parameters for depositing other materials generally will be the same or similar to these conditions, but need not be identical to the parameters disclosed herein.

| | | E-beam | | | | | |
|---|---|---|---|---|---|---|---|
| Coating | Crucible | high | | Deposition | | | |
| Material | material | voltage | Emission | Rate | Ramp 1 | Ramp 2 | Ramp 3 |
| Silicon | Copper | 10 kV | 400 mA | 0.5 nm/s | 20 s/38% | 40 s/46% | 40 s/51% |

| Hold | | Arc | Anode-to Ground | | |
|---|---|---|---|---|---|
| Power | Arc Current | Voltage | Voltage | Plasma Gas | Reaction Gas |
| 22.0% | 55 A | 55 V | 35 V | Argon at 2.8 mbar | Oxygen at 1.0 × 10⁻³ mbar |
| | | | | within plasma | within coating vessel |

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. An optical window for use within an endoscope, comprise:
a substrate sized to be coupled to the endoscope and defining a first surface and at least a second surface; and
at least one coating applied to at least one of the first surface and second surface.

2. The optical window of claim 1 wherein the substrate is manufactured from sapphire.

3. The optical window of claim 1 wherein the substrate is manufactured from at least one material selected from the group consisting of doped-sapphire, Al₂O₃, fused silica, glass, composite materials, and silica.

4. The optical window of claim 1 wherein a coating applied to the substrate comprises Hafnium Oxide.

5. The optical window of claim 1 wherein a coating applied to the substrate comprises Silicon Dioxide.

6. The optical window of claim 1 wherein a coating applied to the substrate comprises Aluminum Oxide.

7. The optical window of claim 1 wherein a coating applied to the substrate comprises Tantulum Pentoxide.

8. The optical window of claim 1 wherein a coating applied to the substrate is selected from the group consisting of silicon, titanium, aluminum, tantalum, hafnium, zirconium, anti-reflective coatings, bandpass filter coatings, wavelength selective coatings, and protective overcoats.

9. An endoscope window configured to withstand multiple autoclaving processes, comprising:
a sapphire substrate sized to coupled to an endoscopy handpiece and defining a first surface and at least a second surface;
at least one first surface coating layer applied to the first surface; and
at least one second surface coating applied to the second surface.

10. The device of claim 9 wherein the first surface coating and second surface coating are the same.

11. The device of claim 9 wherein the first surface coating and second surface coating are different.

12. The device of claim 9 wherein the at least one of the first surface coating and second surface coating comprises Hafnium Oxide.

13. The device of claim 9 wherein the at least one of the first surface coating and second surface coating comprises Silicon Oxide.

14. The device of claim 9 wherein the at least one of the first surface coating and second surface coating comprises Aluminum Oxide.

15. The device of claim 9 wherein the at least one of the first surface coating and second surface coating comprises Tantulum Pentoxide.

16. The device of claim 9 wherein the at least one of the first surface coating and second surface coating is selected from the group consisting of silicon, titanium, aluminum, tantalum, hafnium, zirconium, anti-reflective coatings, bandpass filter coatings, wavelength selective coatings, and protective overcoats.

17. A method of producing a coated endoscope window configured to withstand multiple autoclaving processes, comprising:
positioning one or more endoscope window substrates within an evacuatable coating vessel;
inserting at least one coating material into at least one containment structure positioned located within the coating vessel;
evacuating the containment vessel to a about pressure of about 3×10−6 mbar or less;
activating one or more intense electron beams into the containment structure
vaporizing the coating material at about room temperature or greater with the electron beam; and
depositing the vaporized coating material onto the one or more endoscope windows.

18. A method of producing a coated endoscope window configured to withstand multiple autoclaving processes, comprising:
positioning one or more endoscope window substrates within an evacuatable coating vessel having one or more coating material located therein;
inserting at least one coating material into at least one containment structure positioned located within the coating vessel; and
depositing the vaporized coating material onto the one or more endoscope windows at room temperature using an ion plating process.
